(19) United States
(12) Patent Application Publication
(10) Pub. No.: US 2025/0166795 A1
(43) Pub. Date: May 22, 2025

(54) SMART ANNOTATION TOOL FOR PATHOLOGICAL STRUCTURES IN BRAIN SCANS

(71) Applicant: KONINKLIJKE PHILIPS N.V., EINDHOVEN (NL)

(72) Inventors: ARNE EWALD, HAMBURG (DE); NICK FLAESCHNER, HAMBURG (DE); FABIAN WENZEL, HAMBURG (DE); WILLEM HUIJBERS, TILBURG (NL); MARCO MATTERS, VALKENSWAARD (NL); ROLF MATHIAS JOHANNES NICOLAAS LAMERICHS, LIEMPDE (NL)

(21) Appl. No.: 18/832,959

(22) PCT Filed: Jan. 19, 2023
(86) PCT No.: PCT/EP2023/051193

§ 371 Date: Jul. 25, 2024

Related U.S. Application Data
(60) Provisional application No. 63/303,046, filed on Jan. 26, 2022.

(51) Int. Cl. G16H 30/40; G06T 7/11

(52) U.S. Cl. CPC G16H 30/40; G06T 7/11; G06T 2207/10072; G06T 2207/30016

(57) ABSTRACT

A method for analyzing two medical images, including: receiving a first image along with weak annotation of the first image; receiving a second image; transferring the weak annotations to the second image; registering the first image to the second image based upon the weak annotations on the first image and the second image to produce registration parameters; aligning the received first image and received second image using the registration parameters; and retransferring the weak annotation to the second aligned image.

Example embodiments disclosed herein relate to processing medical image information.

### BACKGROUND

White matter hyperintensities are bright spots that appear in brain scans (e.g., T2-weighted MRIs). These spots are caused by small lesions or other structures that adversely affect patient health. The number, size, and evolution of brain structures (especially over time) may serve as biomarkers for various pathologies (e.g., muscular sclerosis, stroke, dementia, hepatic encephalopathy, general aging effects), and in some cases may serve as neuroimaging markers of brain frailty. For these reasons, annotating brain scans to determine the development of new structures or the progression of existing ones may have clinical significance in the care and treatment of patients.

Annotating structures in brain scans is currently being performed manually. These methods are tedious and subject to error, often depending on the skill of the radiologist. Even when performed by an experienced professional, important information can be overlooked, including those that could play a role in assessing the condition of a patient. Further, lesions in patients may be evaluated over time to determine changes in the lesions, and performing this manually can be challenging for a radiologist.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

A method for analyzing two medical images, including: receiving a first image along with weak annotation of the first image; receiving a second image; transferring the weak annotations to the second image; registering the first image to the second image based upon the weak annotations on the first image and the second image to produce registration parameters; aligning the received first image and received second image using the registration parameters; and retransferring the weak annotation to the second aligned image.

Various embodiments are described, further including analyzing the second and/or first image.

Various embodiments are described, wherein analyzing the second and/or first image includes subtracting the first aligned image and the second aligned image and further including: displaying the subtracted images where positive and negative results are displayed differently.

Various embodiments are described, wherein analyzing the second and/or first image includes comparing a region associated with a weak annotation in the first aligned image and the second aligned image.

Various embodiments are described, wherein analyzing the second and/or first image includes segmenting a region associated with a weak annotation in the second altered image.

Various embodiments are described, wherein analyzing the second and/or first image includes further segmenting a region associated with the weak association in the first altered image, and comparing the segmented regions associated with the weak annotations associated with the first image with those in the second image.

Various embodiments are described, wherein analyzing the second and/or first image includes segmenting a region associated with a weak annotation in the second altered image in two dimensions and propagating the segmentation to images of adjacent slices resulting in a three dimensional segmentation.

Various embodiments are described, further including altering the regions associated with the weak annotations in the first image and the second image to produce an altered first image and an altered second image, wherein registering the first image to the second image is further based upon first altered image and the second altered image.

Various embodiments are described, wherein altering the regions associated with the weak annotations in the first image and the second image includes removing the regions associated with the weak annotations from the first image and the second image.

Various embodiments are described, wherein altering the regions associated with the weak annotations in the first image and the second image includes down-weighting the regions associated with the weak annotations from the first image and the second image.

Various embodiments are described, wherein altering the regions associated with the weak annotations in the first image and the second image includes applying a generative adversarial network (GAN) to the regions associated with the weak annotations from the first image and the second image.

Further various embodiments relate to a system configured to analyze two medical images, including: a memory; a processor coupled to the memory, wherein the processor is further configured to: receive a first image along with weak annotation of the first image; receive a second image; transfer the weak annotations to the second image; register the first image to the second image based upon the weak annotations on the first image and the second image to produce registration parameters; align the received first image and received second image using the registration parameters; and retransfer the weak annotation to the second aligned image.

Various embodiments are described, where in the process is further configured to analyze the second and/or first image.

Various embodiments are described, wherein analyzing the second and/or first image includes subtracting the first aligned image and the second aligned image and further including: displaying the subtracted images where positive and negative results are displayed differently.

Various embodiments are described, wherein analyzing the second and/or first image includes comparing a region associated with a weak annotation in the first aligned image and the second aligned image.

Various embodiments are described, wherein analyzing the second and/or first image includes segmenting a region associated with a weak annotation in the second altered image.

Various embodiments are described, wherein analyzing the second and/or first image includes further segmenting a region associated with the weak association in the first altered image, and comparing the segmented regions associated with the weak annotations associated with the first image with those in the second image.

Various embodiments are described, wherein analyzing the second and/or first image includes segmenting a region associated with a weak annotation in the second altered image in two dimensions and propagating the segmentation to images of adjacent slices resulting in a three dimensional segmentation.

Various embodiments are described, wherein the processor is further configured to alter the regions associated with the weak annotations in the first image and the second image to produce an altered first image and an altered second image, wherein registering the first image to the second image is further based upon first altered image and the second altered image.

Various embodiments are described, wherein altering the regions associated with the weak annotations in the first image and the second image includes removing the regions associated with the weak annotations from the first image and the second image.

Various embodiments are described, wherein altering the regions associated with the weak annotations in the first image and the second image includes down-weighting the regions associated with the weak annotations from the first image and the second image.

Various embodiments are described, wherein altering the regions associated with the weak annotations in the first image and the second image includes applying a generative adversarial network (GAN) to the regions associated with the weak annotations from the first image and the second image.

### BRIEF DESCRIPTION OF THE DRAWINGS

Additional objects and features of the invention will be more readily apparent from the following detailed description and appended claims when taken in conjunction with the drawings. Although several example embodiments are illustrated and described, like reference numerals identify like parts in each of the figures, in which:

FIG. 1 illustrates an embodiment of a system for processing medical images;

FIG. 2 illustrates an embodiment of a method for processing medical image;

FIG. 3 illustrates an example of weak annotations on a medical image;

FIG. 4 illustrates an example of a mask;

FIG. 5 illustrates an embodiment of image segmentation logic;

FIG. 6 illustrates an embodiment of a voxel classification network;

FIG. 7 illustrates an embodiment of a voxel classification network;

FIG. 8 illustrates an embodiment of a method for generating a 3D bounding box for a brain scan;

FIG. 9A illustrates an example of an annotated at a first time, and FIG. 9B illustrates an example of an annotated and segmented image at a second time; and

FIG. 10 illustrates a method for improving the registration of two images taken at different times.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

Semantic Segmentation is a computer vision process that extracts features of an image and then groups pixels into classes that correspond to those features. Once generated, the pixels of each class may be separated (or otherwise distinguished) from pixels in other classes through the use of a segmentation mask. After an image is segmented in this manner, it may be processed using an artificial intelligence (or machine-learning) model such as an voxel classification network.

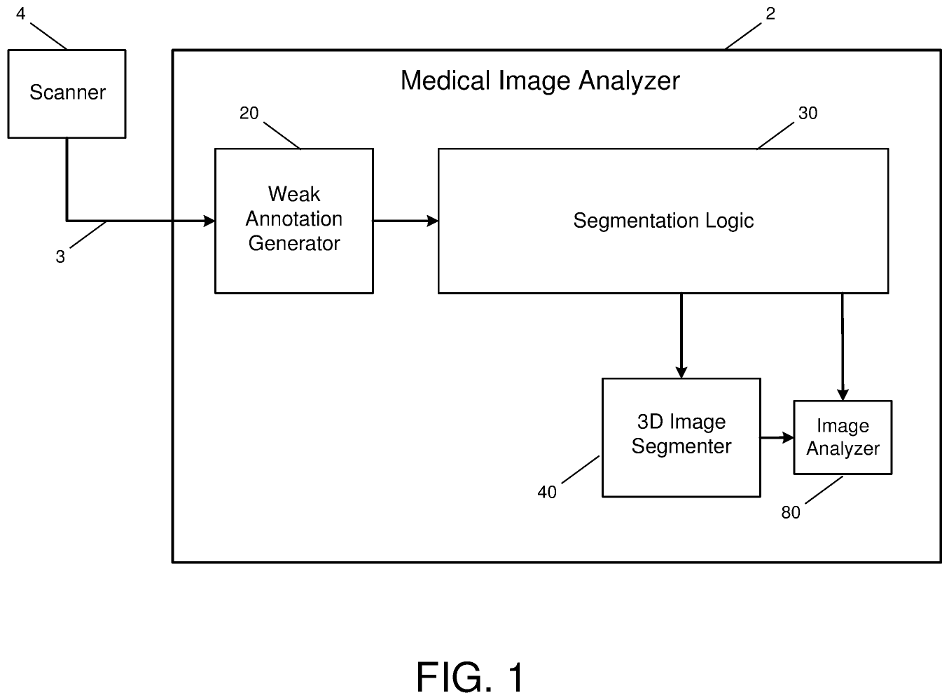

FIG. 1 illustrates an embodiment of a system 1 including a medical image analyzer 2 for processing medical images such as, but not limited to, brain scan images. The medical image analyzer may be coupled to receive baseline images 3 from an imaging system 4, which, for example, may be a magnetic resonance imaging (MRI) scanner, a computed tomography (CT) scanner, or another imaging system. The baseline images may correspond, for example, to a series of image slices of the brain of a patient under evaluation.

Referring to FIG. 1, the medical image analyzer 2 may include a weak annotation generator 20 and image segmentation logic 30. The weak annotation generator may include a processing system implemented as a software tool which generates weak annotations for one or more of the image slices. In one embodiment, the weak annotations may be generated as object-level labels overlaid the image slices. As will be discussed in greater detail below, the object-level labels may include bounding boxes which, for example, may be generated manually by a physician or radiologist or automatically by image annotation software.

The image segmentation logic 30 that performs image segmentation on one or more regions of interest that have been weakly annotated by each of the bounding boxes generated for an image slice. The annotations may be used to determine a mask for region(s) of interest delineated by the bounding box(es).

In one embodiment, the segmentation may be performed using an artificial intelligence (AI) model classifier, such as, but not limited, to a U-Net classifier as described in detail below. The AI model classifier may produce improved segmentations of the image slices in a way that allows for more effective identification and analysis of features in the brain that directly correlate to the condition of the patient. Other image analysis tools may also be used to segment the region of interest.

In addition to the foregoing features, the medical image analyzer 2 may also include a three-dimensional (3D) image segmenter 40 which propagates and two-dimensional (2D) mask from one image slice onto an adjacent image slice and then refining the resulting 3D segmentation using a machine learning model, to generate a 3D segmentation of one or more structures (e.g., lesions) in the brain scan. This may allow for a determination of the growth, extent and/or other characteristics relating to these structures, not only in lateral directions along the x and y axes but also along the z-axis. The 3D segmentation therefore provides a volumetric indication of the brain structure(s) of interest, which may lead to an improved understanding of the condition of the patient and the treatment to be applied.

The 3D image segmenter 40 may also generate three-dimensional bounding boxes by extending the bounding boxes (or other types of weak annotations that may be used). The 3D bounding boxes may then be applied, for example, to a 3D image generated by a subsequent brain scan of the same patient in order to determine changes in the structure over time.

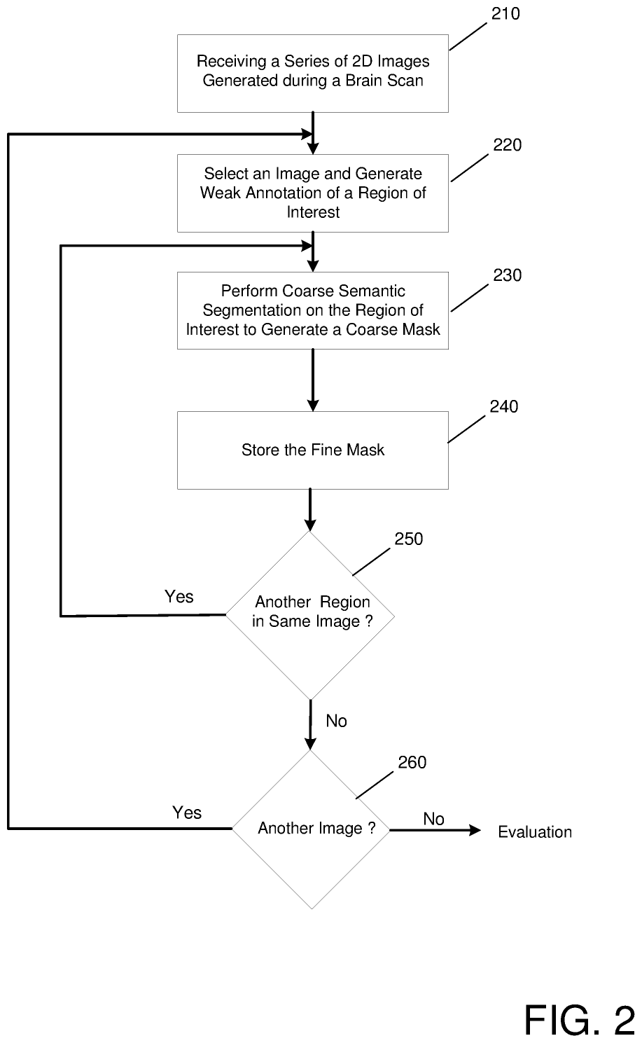

FIG. 2 illustrates an embodiment of a method for processing medical image information. The method may be performed using any of the system embodiments described herein or may be performed using a different system. For illustrative purposes, the method will be described as being performed by the system of FIG. 1 using medical image analyzer 2.

Referring to FIG. 2, the method includes, at 210, receiving a series of two-dimensional images generated during a brain scan of a patient. The images correspond to image slices that result in a 3D image targeting select portions of the brain, which may be used as a basis to assess the condition of the patient suspected of having a stroke or some other disease. As previously indicated, the images may be MRI images, CT images, or ones generated by another type of imaging system. For purposes of discussion, the images will be assumed to be MRI images.

At 220, one image slice is selected which is believed to include one or more structures that provide an indication of patient morbidity. For example, the image slice may include one which shows the middle cerebral artery (MCA) that may have been affected by an ischemic stroke. Once selected, at least one weak annotation is generated for the image slice. The weak annotation may be in the form of an object-level label generated by the weak annotation generator 20 (e.g., an annotation software tool). One example of the weak annotation may include a bounding box that is overlaid (or otherwise designated) on the image slice at a position designated by the physician or automatically determined by a feature extractor. For clinical evaluation purposes, the bounding box may be drawn around a structure that appears, for example, as a bright spot in the image slice. The bright spot may correspond to a white matter hyperintensity (WMH) area of a type that is often associated with a lesion. While WMHs are of interest, the bounding box may be drawn around other structures in the image slice that are different from a structurally formally considered to be a WMH.

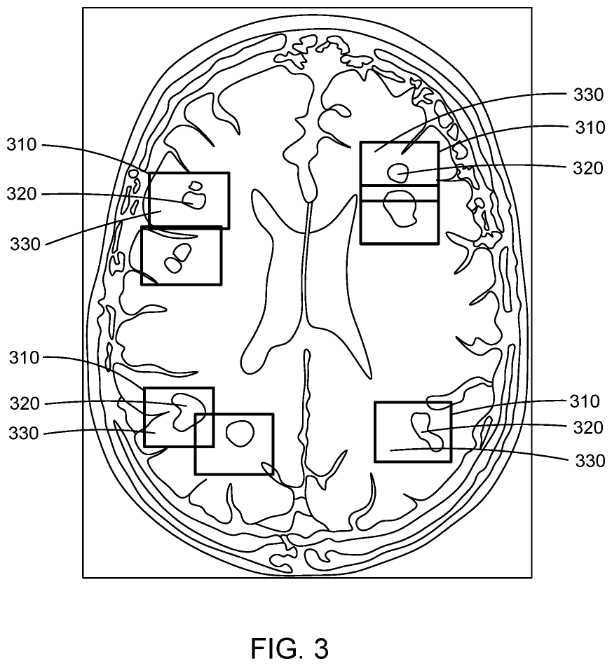

FIG. 3 illustrates an example of an image slice which includes a plurality of structures that appear as bright spot WMHs. When a plurality of structures exist in an image slice, a bounding box may be drawn to encompass one or more of the structures. In some cases, it may be beneficial for each bounding box to include only one structure. However, in other cases, a single bounding box may be generated to encompass a multiple structures, for example, in the case where the multiple structures are separate from one another but very closely spatially related.

In the example illustrated in FIG. 3, weak annotations in the form of bounding boxes 310 are drawn to encompass respective ones of a plurality of structures (bright areas) 320. In this case, the structures are located on both lateral portions (or hemispheres) of the brain. As indicated, these structures (or WMHs) may be lesions may be caused by stroke. In other cases, the structures may be the result of cerebral small vessel disease or may be indicative of another condition, such as but not limited to dementia, Alzheimer's disease, muscular sclerosis, hepatic encephalopathy, general aging effects or other forms of cognitive disfunction.

At 230, a semantic segmentation operation is performed for each of the regions of interest enclosed by the weak annotations. As indicated above, each of the regions of interest (330 in FIG. 3) include at least one structure. (For the balance of this discussion, the weak annotations will be considered to be bounding boxes.) The segmentation performed in operation 230 generates a mask of the structure in the region of interest and may be carried out by an AI model.

The AI model may include Deep Learning Model, such as, but not limited to, a convolutional neural network (CNN). The CNN model may be one which, for example, implements a weakly supervised segmentation of the region of interest. Such a CNN model may be trained with datasets that include pixel regions with one or more bright spots (e.g., WMHs or other brain structures) with surrounding areas that do not correspond to brain structures of interest. The various convolutional layers of the model may process the training datasets to output masks that separate the structures from the surrounding areas. In this way, the model implementing the segmenter may operate as a classifier that, first, recognizes the bright spots from other portions in the regions of interest and, then, extracts (or separates) only those pixels that roughly correspond to those spots. Any other segmentation methods may be used as well, included for example, graph cuts or simple thresholding.

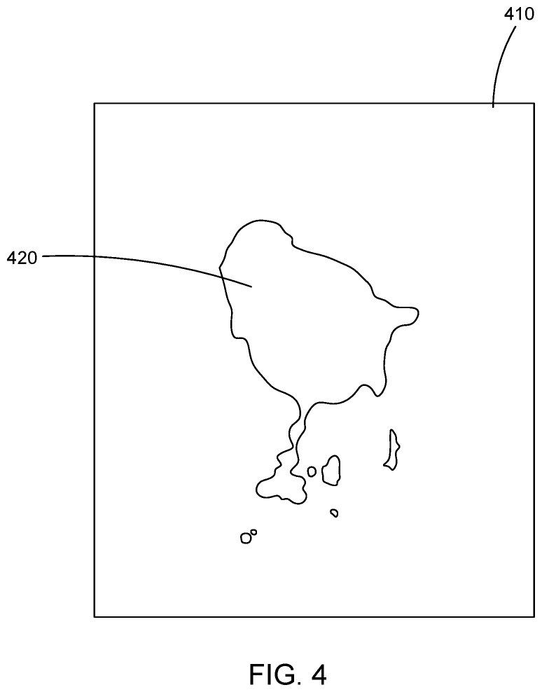

FIG. 4 illustrates an example of a mask 410 of a brain structure that may be generated by the segmentation operation 230. The mask 430 may identify the pixels 420 that only correspond to brain structures that have clinical significance.

In one embodiment, the segmentation operation may be implemented using other methods. For example, because the brain structures of interest appear as bright spots, one embodiment may perform segmentation using a thresholding algorithm, e.g., all pixels having grayscale (or Hounsfield unit (HU) values) above a predetermined threshold may be extracted as those corresponding to a brain structure. In other embodiments, the CNN model may be enhanced by a graph model, but this is not necessary for all applications.

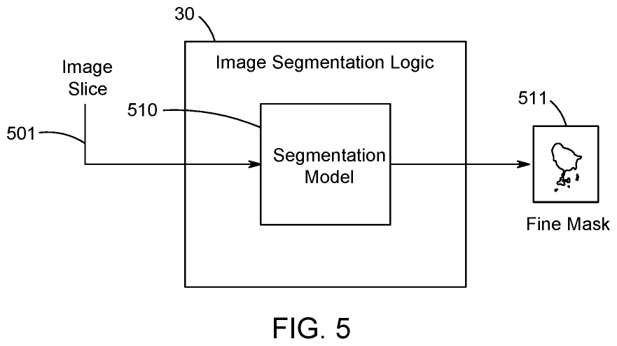

FIG. 5 illustrates an embodiment of the image segmentation logic 20 which includes a segmentation model, for example, as described above. As illustrated in FIG. 5, the segmentation model receives an image slice 501 or a complete 3D image and generates a mask 511 using the segmentation mode 510. To generate the mask, one embodiment of the segmentation model may generate feature vectors from the image slice 501. Once the feature vectors have been generated, the segmentation model may use a number of convolutional layers to generate the mask. For example, the segmentation model may include an initial convolutional layer having a first number of kernels (with predetermined values) and which generate a multi-dimensional vector of a first size. This vector may then be input into at least a second convolutional layer with a second number of kernels that generates another multi-dimensional vector of a second size different from the first size. A subsequent layer of the segmentation model may include a fully connected layer with a predetermined number of input nodes and output nodes respectively representing the class probabilities (structure/no structure) corresponding to a decision indicating whether each pixel in the region of interest (defined by the bounding box) corresponds to a brain structure of interest. It is noted that the specific parameters, number and size of the layers, number of feature maps, number of input dimensions, etc. of the network described above are just examples, and other parameters, number and size of the layers, number of feature maps, number of input dimensions, etc. may be used instead.

In another embodiment, the segmentation classifier used to perform the segmentation may be based on a U-Net architecture. The U-Net may include a contracting path and an expansive path, which gives it the U-shaped architecture. The contracting path may be a convolutional network that applies a repeated application of convolutions, each followed by a rectified linear unit (ReLU) and a max pooling operation. During the contraction, spatial information is reduced while feature information is increased. The expansive pathway combines the feature and spatial information through a sequence of up-convolutions and concatenations with high-resolution features from the contracting path. In one implementation, successive layers may be added which replaces pooling operations with upsampling operators. Hence, the layers of the U-Net increase the resolution of the output, which in this case produces a segmentation of the WMH.

In one embodiment, the U-Net may include a large number of feature channels in the upsampling portion, which allow the network to propagate context information to higher resolution layers. As a consequence, the expansive path is more or less symmetric to the contracting part, and yields a U-shaped architecture. The network may only use the valid part of each convolution without any fully connected layers. To predict pixels in the border region of the image, the missing context may be extrapolated, for example, by mirroring the input portion of the image corresponding to the bounding box.

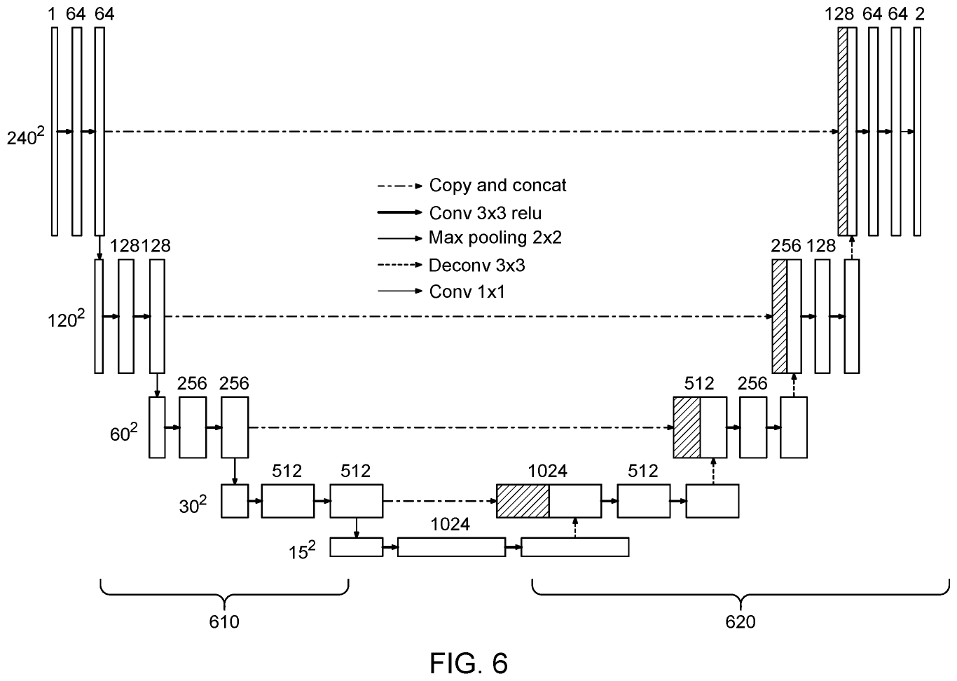

FIG. 6 illustrates an embodiment of a U-Net architecture 600 which may perform the segmentation operation. This architecture includes an encoder 610 implementing a down-sampling path and a decoder 620 implementing an upsampling path. The down-sampling path in the encoder 610 includes a plurality of convolution blocks (e.g., 5 blocks), each block having a number (e.g., 2) convolution layers with a filter of a predetermined size (e.g., 3×3) and a stride having a predetermined value (e.g., 1) in both directions and rectifier activation, which increases the number of feature maps, for example, from 1 to 1024. For the down-sampling, max pooling with a stride (e.g., 2×2) may be applied to the end of every block except the last block. As a result, the size of feature maps decrease from a first value (e.g., 240×240) to a second value (e.g., 15×15).

In the up-sampling path in the decoder 620, every block starts with a de-convolutional layer with a predetermined filter size (e.g., 3× 3) and predetermined stride (e.g., 2× 2), which doubles the size of feature maps in both directions but decreases the number of feature maps, for example, by two. As a result, the size of feature maps may increase from the second value (e.g., 15×15) to the first value (e.g., 240×240). In every up-sampling block, two convolutional layers reduce the number of feature maps of concatenation of de-convolutional feature maps and the feature maps from encoding path. In one embodiment, the U-Net architecture may optionally use zero padding to maintain the output dimension for all the convolutional layers of both downsampling and up-sampling path. Finally, a convolutional layer (e.g., 1×1) may be used to reduce the number of feature maps (e.g., to two) to reflect the foreground and background segmentation, respectively. No fully connected layer may be invoked in the network. Other example parameters of the network may be as follows:

| Parameters | Value |
|---|---|
| Number of convolutional blocks | [4, 5, 6] |
| Number of deconvolutional blocks | [4, 5, 6] |
| Regularization | L1, L2, Dropout |

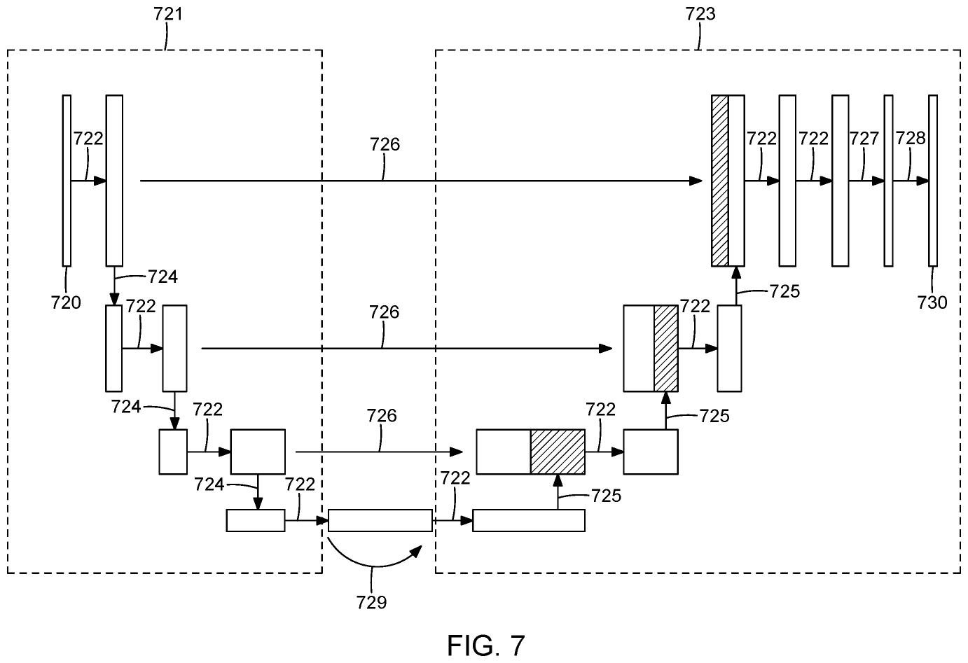

FIG. 7 illustrates another embodiment of a U-Net architecture 700 which may perform the segmentation operation. This architecture includes an encoder 710 implementing a down-sampling path and a decoder 720 implementing an upsampling path. The encoder 710 and decoder 720 are formed from various units 722, 724, 725, 727 and 728. The architecture is a fully convolutional network for deep learning that may accept inputs of any size.

While a few examples of segmentation algorithms have been disclosed, other segmentation algorithms may be used as well.

In FIG. 7, the grey boxes may represent data such as image areas that have been segmented in accordance with the bounding boxes, e.g., may correspond to masks. The segmentation mask may indicate fine or intermediary collections of feature values, and the arrows may represent units or layers. For the convolution or encoder segment 710, convolutional units 722 (e.g., leaky rectified linear units (ReLU)) and max pooling units 724 may be used. For the decoder 720, convolutional units 722 (e.g., ReLU), transposed-convolution units 725 (e.g., ReLU), convolutional units 27 (e.g., Leaky ReLU), and a soft-max unit 728 may be used. Each convolutional (or transposed-convolutional) unit 722, 725, and 727 may contain a batch normalization layer and a ReLU activation, followed by a 3×3×3 or other size convolutional layer. The U-Net architecture may be modified in various ways, for example, more or fewer units 722 and 727 may provided in the decoder to generate the output segmentation for the least level of abstraction or compression.

In operation, the encoder 710 outputs features (or values for features) of the mask to the decoder 23. Bridging units may be used such as treating the units 722 at the greatest level of abstraction as separate from the encoder segment 721 and/or the decoder segment 723.

Other connections than at the bottom of the U-Net architecture (e.g., at the greatest level of abstraction) between the encoder 710 and the decoder 720 may be provided. Connections between different parts of the architecture at a same level of abstraction may be used. At each abstraction level of the decoder, the feature abstraction matches the corresponding encoder level. For example, the feature values output from each convolutional unit 722, in addition to the final or greatest compression of the encoder 710, may be output to the next max-pooling unit 724 as well as to a convolutional unit 722 of the decoder with a same level of abstraction.

The arrows 726 show this concatenation as skip connections which may skip one or more units. The skip connections at the same levels of abstraction may be free of other units or may include other units. Other skip connections from one level of abstraction to a different level of abstraction may be used. In one embodiment, no skip connections between the encoder 710 and the decoder 720 (other than connections at the bottom (e.g., greatest level of abstraction)) may be provided between the encoder 710 and the decoder 720.

In addition to the foregoing features, the U-Net architecture of FIG. 7 may include a long short-term memory (LSTM) unit 729 located at the skip connection 726 at the bottom level of the U-Net. The LSTM unit 729 may, for example, be implemented as a SoftMax layer

The LSTM unit 29 may operate on the values of features at a greatest level of compression. In one embodiment, the LSTM unit 29 is a recurrent neural network (RNN) structure for modeling dependencies over time. In addition to relating spatial features to the output segmentation, temporal features may be included. The variance over time of pixels, voxels, or groups thereof is accounted for by the LSTM unit 29. The values of the features derived from the pixels, voxels or groups thereof may be different for different masks. Thus, the LSTM unit 29 may be positioned to receive feature values and may derive values for the features based on the variance over time or differences over time (e.g., state information) of the input feature values for each node.

In one embodiment, the convolutional LSTM unit 729 may operate may receive the output of the encoder segment 710 and may pass the results to the decoder 720. At the end of the encoder 710, the network has extracted the most compressed features carrying global context. Thus, the convolutional LSTM unit 729 may be positioned at the bottom level of network to extract global features that capture the temporal changes observed over time. In one embodiment, the output from the encoder 710 may skip the LSTM unit 729 so that the decoder 720 receives both the output of the LSTM unit 729 and the output of the encoder 710.

During training, in order to learn to determine patterns over time of the values of features, the LSTM unit 729 may use the spatiotemporal features. For example, the encoder 710 derive values for spatial features in each mask in a sequence. The period over which the patterns are derived may be learned and/or set.

The output of the segmentation classifier (e.g., the U-Net architecture) 20 may correspond to a segmentation mask of the brain structure (e.g., the WMH or bright spot) encompassed within the weak annotation (e.g., bounding box) overlaid on the baseline image of the image slice 501. The segmentation generates a mask 511 which provides an accurate representation of the brain structure of interest in the bounding box. FIG. 4 illustrates an example mask 450 output by the segmentation classifier that identifies the lesion in the bounding box.

The segmentation classifier may also include a two step process, where a first coarse segmentation is done with a coarse resolution to segment the WMH inside the bounding box. Then this may be resampled to a finer resolution and then this image segmented again using the coarse segmentation as an additional input.

At 240, the brain structure represented by the mask may be stored in a database or other storage device for further processing (e.g., 3D segmentation) and/or comparison to an image of the same patient taken at a later time.

At 250, a determination is made as to whether an additional region of interest in the same image slice is to be segmented. If so, operations 220 to 240 may be repeated for the same the additional region of interest, which may included another brain structure in that image slice. If all of the regions of interest in the image slice have been segmented, operation 260 is performed.

At 260, a determination is made as to whether the next image (e.g., image slice) of the series of 2D images received in operation 210 are to be segmented. If the next image (e.g., image slice) in the series of 2D images are to be segmented, then process follow returns to operation 210 for generating masks for one or more structures the next image slice. If there is not a next image (e.g., image slice) in the series of 2D images to be segmented, then the segmented images (e.g., the masks) stored for the brain scan of the patient may be provided for further processing or evaluation.

The further processing may be performed by another Deep Neural Network which, for example, may classify the brain structure(s) in the mask(s). In another embodiment, the further processing may include generating a 3D segmentation (with or without forming corresponding 3D bounding boxes) for the brain structure(s), e.g., masks, generated for the input images. In another embodiment, the segmentations may be output for review by a physician or radiologist for purposes of determining, for example, one or more treatment options.

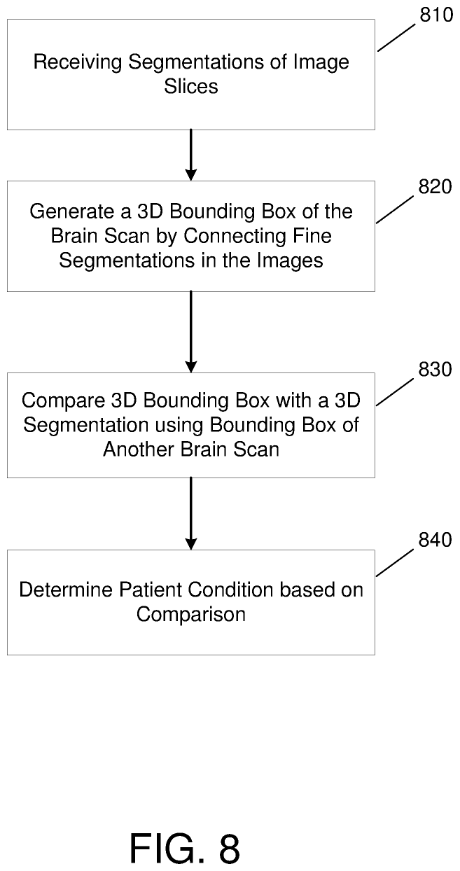

FIG. 8 illustrates an embodiment of a method for generating a 3D bounding box for a brain scan. The method includes, at 810, receiving segmentations (e.g., masks) for different image slices of a brain scan of a patient. The segmentations may be generated by the method of FIG. 2 or another method. When generated by the method of FIG. 2, the segmentations may be retrieved from a database or other storage device. In one embodiment, the segmentations received in operation 810 may be adjacent image slices or non-adjacent image slices, provided that the segmentations correspond to the same one or more brain structures for which the segmentations were generated.

At 820, a 3D bounding box is generated for each structure. This may be accomplished by extending the segmentations (e.g., masks) corresponding to the same structures generated for consecutive ones of the image slices. By extending the segmentation across multiple (2D) image slices (e.g., in the ±z-direction), a 3D bounding box for each structure may be generated. Such an extension may involve, for example, registering the image slices (containing the segmentations) relative to one another or to a reference so that the images slices are properly aligned. Then, each slice may be refined, for example, using the input from the prior slice as an additional channel. As a result, a full 3D bounding box may be generated for each structure of interest in the patient brain scan.

Additional operation may include, at 830, comparing the image with the 3D bounding box with a later captured brain scan. Then, an additional operation may include, at 840, comparing segmented images to determine how the condition of the brain of the patient has changed over time. An example may be explained as follows.

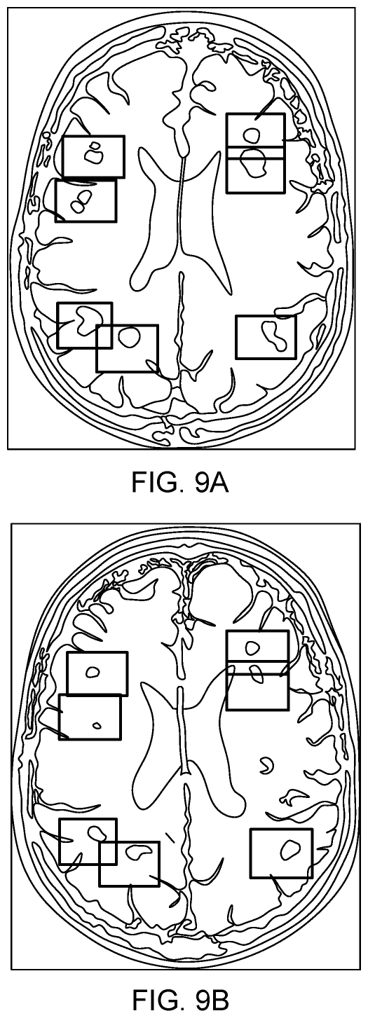

FIGS. 9A and 9B illustrate a side-by-side comparison of images taken over time. For example, the weak annotation performed relative to the image slice of FIG. 9A may be considered to have been take at time t=t₀. At some subsequent time t=t₁, a second brain scan may be performed to obtain the same type of image slice to be used for comparison, as indicted in FIG. 9B. The differences between times t₀ and t₁ may be any span of time (e.g., days, weeks, months, years) for which patient assessment is to be performed.

In one embodiment, the system may include a subtractor (e.g., 80 in FIG. 1) which is configured to generate a subtraction image by subtracting the segmented image at t₁ from the segmented image at to. This may allow differences in lesion size to be determined, as well as other features which mark a change in the state of the WMH lesion or progression of a corresponding disease. These differences may be automatically quantified, for example, by applying a thresholding algorithm to the subtraction image, which may define a 3D bounding box for new lesions, even for imperfect registrations. Further the subtract images identifies area where there are changes that may guide physician or radiologist to look for changes in the areas where the difference is greatest.

In one embodiment, color-overlays may be generated to illustrate where the images are different. The color scheme may be different according to sign, e.g., for shrinking or growing structures. Also, in one case, transparency and blending after registration and overlay may be performed. Further, the bounding boxes from the first image may be placed on the difference image and the second image to be able to easily compare areas.

In order to compare these image slices, an initial registration operation may be performed. Various know registration methods are known and may be used. An issue arises when the entire image at t₁ is to be registered with the entire image at to. Because of changes in the WMH regions between the two scans, the registration may difficult and have limited accuracy. An approach to improve the accuracy of the registration will now be described that improve the ability to subtract two images from one another.

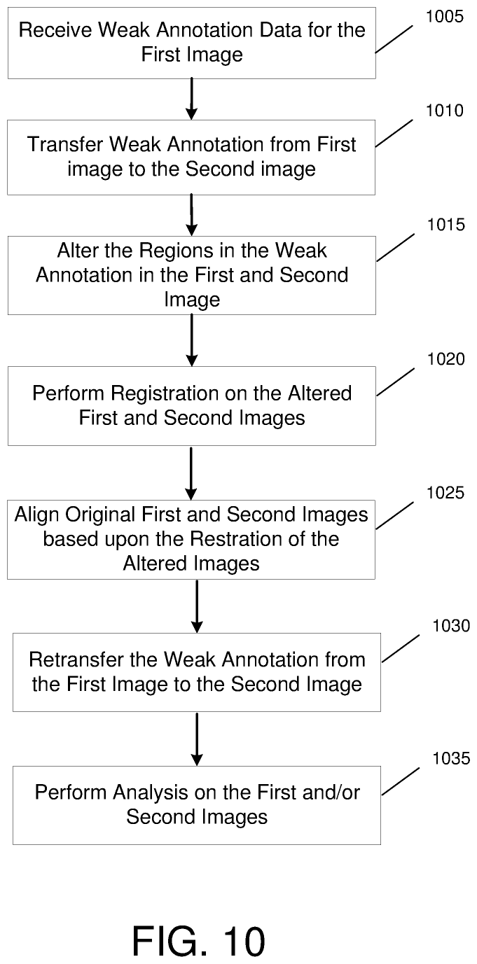

FIG. 10 illustrates a method for improving the registration of two images taken at different times. At 1005, weak annotations or bounding box data are received for a first image. At 1010, the bounding boxes placed on the first image are transferred to the second image. This may be just a direct transfer, or some of the known registration methods may be used to assist in this transfer. At 1015, the regions inside the bounding boxes in both images may be altered. This may be done in the following different ways.

In one embodiment, the regions within the bounding boxes of the scans may be altered by removing them from the images or down-weighting these areas.

In another embodiment, the regions within the bounding boxes of the scans may be altered by applying a style generative adversarial network (GAN) in the areas within the bounding boxes. The GAN may be a trained to replace the regions in the bounding boxes with healthy looking brain tissue in the areas. As a result, the GAN will replace the image area inside the bounding boxes with a healthy looking image representing brain tissue. This should be done similarly for both the first and second scans, so that corresponding areas in the bounding boxes in each of the images will now appear to be similar. This alteration will help improve the registration process that follows. In another embodiment, the whole image may be run through the GAN to replace lesions with healthy tissue.

The GAN may be trained by inputting healthy images as inputs. Then when images with lesions are input, the output images will replace the lesions with simulated healthy tissue.

Alternatively, the images may not be altered, and the images with the weak annotations are used to perform the registration.

At 1020, the method performs a registration algorithm on the first and second altered images. Because the lesion areas have been removed or altered, this registration process will provide better results because the effects of changing lesions have been removed. As discussed above, any type of known registration algorithm may be used. The registration algorithm will provide output paraments regarding how to align the first and second altered images. At 1025, these registration output parameters may be used to align the original first and second images. Because the lesions were removed during the registration process, this should result in a better registration versus applying the registration algorithm directly on the original images. At 1030, now that the original images are aligned, the received bounding boxes may be retransferred to the second image, and because of the improved registration they will be more accurately placed on the second image.

At 1035, further analysis of the first and/or second images may be performed. This analysis may include a full segmentation of the first and/or second images. At this point a subtraction of the two images will provide better results indicating the changes between the two images. Both the first and second images with the bounding boxes may be shown to a physician or radiologist to highlight the areas of interest and to compare the contents of the bounding box in the two images. Also, the bounding boxes may be transferred to a subtraction image, which again will highlight the areas where changes are expected. If there are no changes in the bounding box in the subtraction image that means that there is no significant changes the lesion in the bounding box. Further, of analysis tools may be applied to the contents of the bounding boxes to measure a change in size of the lesions, for example, using segmenting to isolate the lesions in the images. Further, the images may be processed to produce 3D bounding boxes that may be analyzed. Any other beneficial analysis tool may be applied to images after registration and retransfer of the bounding boxes.

Based on comparing FIGS. 9A and 9B, it is apparent that the white spots in the t₁ image are smaller than the white spots generated in the masks in the to image. This indicates a marked improvement in the condition of the patient as a result of treatment. The bounding boxes in the two images highlight areas of interest that can easily be compared, and it can be done so accurately because bounding boxes have been accurately transferred to the new image. In other cases, segmentation algorithms may be applied to corresponding bounding boxes, and the results compared automatically or manually to determine if the lesions have changes. Through this process, empirical evidence may be obtained as confirmation of patient status. This is the case, even when there are lesions that affect the accuracy of the registration process.

In one embodiment, a cascaded approach may be taken to generating segmentations. This may involve performing a resampling with respect to the annotated bounding box. For example, the bounding boxes drawn by a physician will likely differ in size. In this case, each bounding box may be resampled to a fixed size with a coarse image resolution. Then, the structure (e.g., WMH) in the box may be segmented and then resampled to a finer resolution and segmented again to provide the coarse segmentation as an additional input.

In accordance with one or more of the aforementioned embodiments, the methods, processes, and/or operations described herein may be performed by code or instructions to be executed by a computer, processor, controller, or other signal processing device. The computer, processor, controller, or other signal processing device may be those described herein or one in addition to the elements described herein. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods described herein.

Also, another embodiment may include a computer-readable medium, e.g., a non-transitory computer-readable medium, for storing the code or instructions described above. The computer-readable medium may be a volatile or non-volatile memory or other storage device, which may be removably or fixedly coupled to the computer, processor, controller, or other signal processing device which is to execute the code or instructions for performing the operations of the system and method embodiments described herein.

The processors, systems, controllers, segmenters, generators, labelers, simulators, models, networks, scalers, and other signal-generating and signal-processing features of the embodiments described herein may be implemented in logic which, for example, may include hardware, software, or both. When implemented at least partially in hardware, the processors, systems, controllers, segmenters, generators, labelers, simulators, models, networks, scalers, and other signal-generating and signal-processing features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the processors, systems, controllers, segmenters, generators, labelers, simulators, models, networks, scalers, and other signal-generating and signal-processing features may include, for example, a memory or other storage device for storing code or instructions to be executed, for example, by a computer, processor, microprocessor, controller, or other signal processing device. The computer, processor, microprocessor, controller, or other signal processing device may be those described herein or one in addition to the elements described herein. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Although the various exemplary embodiments have been described in detail with particular reference to certain exemplary aspects thereof, it should be understood that the invention is capable of other example embodiments and its details are capable of modifications in various obvious respects. As is apparent to those skilled in the art, variations and modifications can be affected while remaining within the spirit and scope of the invention. The embodiments may be combined to form additional embodiments. Accordingly, the foregoing disclosure, description, and figures are for illustrative purposes only and do not in any way limit the invention, which is defined by the claims. The embodiments may be combined to form additional embodiments.

1. A method for analyzing two medical images using a processing device, comprising:
receiving a first image along with weak annotation of the first image;
receiving a second image;
transferring the weak annotations to the second image;
registering the first image to the second image based upon the weak annotations on the first image and the second image to produce registration parameters;
aligning the received first image and received second image using the registration parameters; and
retransferring the weak annotation to the second aligned image.

2. The method of claim 1, further comprising analyzing the second and/or first image.

3. The method of claim 2, wherein analyzing the second and/or first image includes subtracting the first aligned image and the second aligned image and further comprising:
displaying the subtracted images where positive and negative results are displayed differently.

4. The method of claim 2, wherein analyzing the second and/or first image includes comparing a region associated with a weak annotation in the first aligned image and the second aligned image.

5. The method of claim 2, wherein analyzing the second and/or first image includes segmenting a region associated with a weak annotation in the second altered image.

6. The method of claim 5, wherein analyzing the second and/or first image includes further segmenting a region associated with the weak association in the first altered image, and comparing the segmented regions associated with the weak annotations associated with the first image with those in the second image.

7. The method of claim 2, wherein analyzing the second and/or first image includes segmenting a region associated with a weak annotation in the second altered image in two dimensions and propagating the segmentation to images of adjacent slices resulting in a three dimensional segmentation.

8. The method of claim 1, further comprising altering the regions associated with the weak annotations in the first image and the second image to produce an altered first image and an altered second image, wherein registering the first image to the second image is further based upon first altered image and the second altered image.

9. The method of claim 8, wherein altering the regions associated with the weak annotations in the first image and the second image includes: removing the regions associated with the weak annotations from the first image and the second image, down-weighting the regions associated with the weak annotations from the first image and the second image; and/or applying a generative adversarial network (GAN) to the regions associated with the weak annotations from the first image and the second image.

10. (canceled)

11. (canceled)

12. A system configured to analyze two medical images, comprising:
a memory;
a processor coupled to the memory, wherein the processor is further configured to:
receive a first image along with weak annotation of the first image;
receive a second image;
transfer the weak annotations to the second image;
register the first image to the second image based upon the weak annotations on the first image and the second image to produce registration parameters;
align the received first image and received second image using the registration parameters; and
retransfer the weak annotation to the second aligned image.

13. The system of claim 12, where in the process is further configured to analyze the second and/or first image.

14. The system of claim 13, wherein analyzing the second and/or first image includes subtracting the first aligned image and the second aligned image and further comprising:
displaying the subtracted images where positive and negative results are displayed differently.

15. The system of claim 13, wherein analyzing the second and/or first image includes comparing a region associated with a weak annotation in the first aligned image and the second aligned image.

16. The system of claim 13, wherein analyzing the second and/or first image includes segmenting a region associated with a weak annotation in the second altered image.

17. The system of claim 16, wherein analyzing the second and/or first image includes further segmenting a region associated with the weak association in the first altered image, and comparing the segmented regions associated with the weak annotations associated with the first image with those in the second image.

18. The system of claim 13, wherein analyzing the second and/or first image includes segmenting a region associated with a weak annotation in the second altered image in two dimensions and propagating the segmentation to images of adjacent slices resulting in a three dimensional segmentation.

19. The system of claim 9, wherein the processor is further configured to alter the regions associated with the weak annotations in the first image and the second image to produce an altered first image and an altered second image, wherein registering the first image to the second image is further based upon first altered image and the second altered image.

20. The system of claim 19, wherein altering the regions associated with the weak annotations in the first image and the second image includes: —removing the regions associated with the weak annotations from the first image and the second image; down-weighting the regions associated with the weak annotations from the first image and the second image; and/or includes applying a generative adversarial network (GAN) to the regions associated with the weak annotations from the first image and the second image.

21. (canceled)

22. (canceled)

23. A non-transitory computer readable medium storing instructions than when executed by a processing device cause the processing device to perform the method of claim 1.
